(19) United States
(12) Patent Application Publication
(10) Pub. No.: US 2006/0189682 A1
(43) Pub. Date: Aug. 24, 2006

(54) Water soluble prodrugs of COX-2 inhibitors

(76) Inventors: Joseph E. Payne, Oceanside, CA (US); Steve Poon, So. Pasadena, CA (US); Nicholas D. Smith, San Diego, CA (US); Nicholas S. Stock, San Diego, CA (US); Angela R. McGuire, East Palo Alto, CA (US)

Correspondence Address: MERCK AND CO., INC, P O BOX 2000, RAHWAY, NJ, 07065-0907, US

(21) Appl. No.: 11/345,694

(22) Filed: Feb. 2, 2006

Related U.S. Application Data
(60) Provisional application No. 60/649,188, filed on Feb. 2, 2005.

(51) Int. Cl. A61K 31/343; C07D 493/10

(52) U.S. Cl. 514/462; 549/334

(57) ABSTRACT

Disclosed are water soluble compounds which are useful as prodrugs of COX-2 inhibitors, and pharmaceutical compositions comprising them.

### CROSS REFERENCE TO RELATED APPLICATIONS

This application claims priority under 35 U.S.C. §119(e) to U.S. provisional patent application Ser. No. 60/649,188, filed Feb. 2, 2005.

### FIELD OF THE INVENTION

The invention is directed to water soluble compounds which are useful as prodrugs of COX-2 inhibitors, and pharmaceutical compositions comprising the compounds of the invention. The invention is also directed to methods of treating patients for cyclooxygenase-mediated diseases, including stroke, by administering to the patient a compound or pharmaceutical composition of the invention.

### BACKGROUND OF THE INVENTION

Cyclooxygenase (COX) is a prostaglandin G/H synthase. Non-steroidal, antiinflammatory drugs (NSAIDs) exert most of their antiinflammatory, analgesic and antipyretic activity through inhibition of prostaglandin G/H synthase.

COX has a constitutive form, COX-1, and an inducible form, COX-2. COX-1 is largely responsible for endogenous basal release of prostaglandins, and hence is important in their physiological functions, such as the maintenance of gastrointestinal integrity and renal blood flow. In contrast, COX-2 is mainly responsible for the pathological effects of prostaglandins, where rapid induction of the enzyme occurs in response to inflammatory agents, hormones, growth factors, and cytokines.

Thus, selective inhibitors of COX-2 have similar antiinflammatory, antipyretic and analgesic properties to conventional NSAIDs, but have a diminished ability to induce some of the mechanism-based side effects. In particular, selective COX-2 inhibitors have a reduced potential for gastrointestinal toxicity, a reduced potential for renal side effects, a reduced effect on bleeding times and possibly a lessened ability to induce asthma attacks in aspirin-sensitive asthmatic subjects.

Particular cyclooxygenase diseases or disorders for which COX-2 inhibitors may be useful include stroke. Stroke is a cerebrovascular event, which occurs when the normal bloodflow to the brain is disrupted, and the brain receives too much or too little blood. Stroke is one of the leading causes of death worldwide, and is also one of the most common causes of neurologic disability.

Ischemic stroke, which is the most common type of stroke, results from insufficient cerebral circulation of blood caused by obstruction of the inflow of arterial blood. Normally, adequate cerebral blood supply is ensured by a system of arteries within the brain. However, various disorders, including inflammation and atherosclerosis, can cause a thrombus, i.e., a blood clot that forms in a blood vessel. The thrombus may interrupt arterial blood flow, causing brain ischemia and consequent neurological symptoms. Ischemic stroke may also be caused by the lodging of an embolus (an air bubble) from the heart in an intracranial vessel, causing decreased perfusion pressure or increased blood viscosity with inadequate cerebral blood flow. An embolus may be caused by various disorders, including atrial fibrillation and atherosclerosis.

A second type of stroke, hemorrhagic stroke, involves a hemorrhage or rupture of an artery leading to the brain. Hemorrhagic stroke results in bleeding into brain tissue, including the epidural, subdural, or subarachnoid space of the brain. A hemorrhagic stroke typically results from the rupture of an arteriosclerotic vessel that has been exposed to arterial hypertension or to thrombosis.

During acute ischemic stroke, i.e., the period from the cerebrovascular event up to 24 hours after the event, the arterial occlusion results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of injury. An area around the infracted core, known as the ischemic penumbra, suffers a delayed and less severe infarct. For example, during acute stroke the penumbra may have a reduction in blood flow of from about 20-40%.

Potent and selective furan-2-one derivative COX-2 inhibitors have been disclosed in U.S. Pat. Nos. 5,733,909, 5,849,943, 5,925,631, 6,020,343 and 6,057,319. The selective COX-2 inhibitor 5(S)-ethyl-3-isopropoxy-4-(4-methanesulfonylphenyl)-5-methyl-5H-furan-2-one, which is disclosed in the '343 patent, has demonstrated a reduction in infarct volume in the middle cerebral artery occlusion (MCAO) stroke model.

For the acute treatment of stroke, it is preferred that the selective COX-2 inhibitors be administered to the patient intravenously. However, the furan-2-one compounds disclosed in the above-referenced patents, including 5(S)-ethyl-3-isopropoxy4-(4-methanesulfonylphenyl)-5-methyl-5H-furan-2-one, have poor water solubility, and are thus unsuitable for intravenous formulation. A water soluble prodrug of 5(S)-ethyl-3-isopropoxy-4-(4-methanesulfonylphenyl)-5-methyl-5H-furan-2-one would facilitate the preparation of intravenous formulations.

### SUMMARY OF THE INVENTION

The invention is directed to water soluble prodrugs of the selective COX-2 inhibitor 5(S)-ethyl-3-isopropoxy-4-(4-methanesulfonylphenyl)-5-methyl-5H-furan-2-one, in the form of compounds of formula (I) 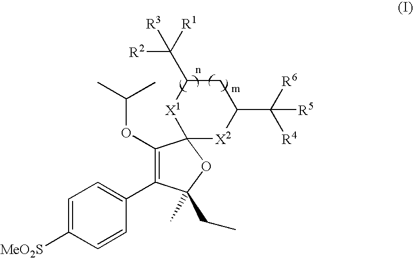
wherein X¹, X², R¹, R², R³, R⁴, R⁵ and R⁶ are as defined below, and pharmaceutically acceptable salts thereof, compounds of formula (II): 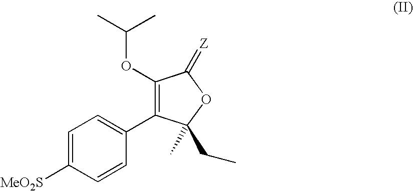
wherein Z is an amino acid or amino acid derivative which is linked at a nitrogen atom to the remainder of compound (II), and pharmaceutically acceptable salts thereof, and compounds of formula (III): 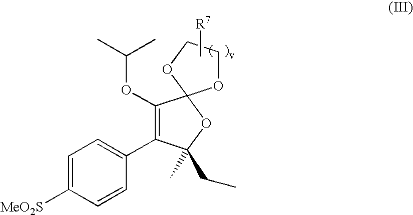
wherein R⁷ is a sugar molecule fused to the remainder of compound (III), and pharmaceutically acceptable salts thereof.

The invention is also directed to pharmaceutical compositions comprising the compounds of formulas (I), (II) and (III), and the use of the compounds and compositions in the treatment of stroke and other COX-2 mediated disorders and diseases.

### DETAILED DESCRIPTION OF THE INVENTION

In one embodiment, the invention is directed to compounds of formula (I) 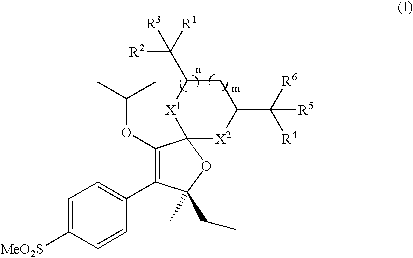
wherein:
X¹ and X² are selected from the group consisting of O, N and S;
n is 0 and m is 1, 2 or 3, or n is 1 and m is 0, 1 or 2;
R¹ and R⁴ are independently selected from the group consisting of
   (1) -Q-R^{a},
   (2) hydroxyl,
   (3) a carbocyclic group having from 3 to 8 ring carbon atoms, optionally having from one to three ring carbon atoms replaced with S, N, C(═O) or O,
   (4) —C₆₋₁₀ aryl, and
   (5) a heteroaryl group having from 5-10 ring atoms,
   wherein said carbocyclic group, aryl and heteroaryl are unsubstituted or substituted with one or more
      (a) halogen,
      (b) cyano,
      (c) —NO₂,
      (d) —C₁₋₆ alkyl, wherein said alkyl is unsubstituted or substituted with one or more halogen,
      (e) —C₁₋₆ alkoxy,
      (f) —C(═O)—(O)_{z}—R^{b},
      (g) —C(═O)—NR^{b}R^{b′},
      (h) —O—C(═O)—R^{b},
      (i) —S(O)_{y}R^{b},
      (j) —S(O)_{y}NR^{b}R^{b′},
      (k) —S(O)_{y}NR^{b}C(═O)C₁₋₆ alkyl, wherein said alkyl is unsubstituted or substituted with one or more halogen,
      (l) —NR^{b}R^{b′},
      (m) —NR^{b}—C(═O)—R^{b′}, and
         y is 0, 1 or 2
         z is 0 or 1;
Q is selected from the group consisting of
   (a) —O—,
   (b) —O—C(═O)—,
   (c) —S—,
   (d) —SO₂—,
   (e) —NR^{b},
   (f) —NR^{b}—C(═O)—, and
   (g) —O—PO₃—;
   R^{a}, R^{b} and R^{b′} are independently selected from the group consisting of:
      (i) —C₁₋₁₀ alkyl,
      (ii) —C₂₋₁₀ alkenyl,
      (iii) —C₂₋₁₀ alkynyl,
      (iv) a carbocyclic group having from 3 to 8 ring carbon atoms, optionally having from one to three ring carbon atoms replaced with S, N, C(═O) or O, and
      (v) —C₆₋₁₀ aryl,
      wherein said carbocyclic group, alkyl, alkenyl, alkynyl and aryl are unsubstituted or substituted with one or more
      (A) halogen,
      (B) cyano,
      (C) —NO₂,
      (D) —C₁₋₆ alkyl, wherein said alkyl is unsubstituted or substituted with one or more halogen,
      (E) —C₁₋₆ alkoxy,
      (F) —C(═O)—(O)_{z}—R^{c},
      (G) —C(═O)—NR^{c}R^{c′},
      (H) —O—C(═O)—R^{c},
      (I) —S(O)_{y}R^{c},
      (J) —S(O)_{y}NR^{c}R^{c′},
      (K) —S(O)_{y}NR^{c}—C(═O)—C₁₋₆ alkyl, wherein said alkyl is unsubstituted or substituted with one or more halogen,
      (L) —NR^{c}R^{c′}, and
      (M) —NR^{c}—C(═O)—R^{c′},
      and R^{c} and R^{c′} are independently selected from the group consisting of
      (1) hydrogen,
      (2) —C₁₋₁₀ alkyl,
      (3) —C₂₋₁₀ alkenyl,
      (4) —C₂₋₁₀ alkynyl,
      (5) a carbocyclic group having from 3 to 8 ring carbon atoms, optionally having from one to three ring carbon atoms replaced with S, N, C(═O) or O,
      (6) —C₀₋₁₀ alkyl-C₆₋₁₀ aryl, and
      (7) a heteroaryl group having from 5-10 ring atoms;
R², R³, R⁵ and R⁶ are independently selected from the group consisting of
   (1) hydrogen,
   (2) —C₁₋₁₀ alkyl,
   (3) —C₂₋₁₀ alkenyl,
   (4) —C₂₋₁₀ alkynyl, or
   (5) —C₆₋₁₀ aryl,
   wherein said alkyl, alkenyl, alkynyl, and aryl are unsubstituted or substituted with one or more
      (a) halogen,
      (b) cyano,
      (c) —NO₂,
      (d) —C₁₋₆ alkyl, wherein said alkyl is unsubstituted or substituted with one or more halogen,
      (e) —C₁₋₆ alkoxy,
      (f) —C(═O)—(O)_{z}—R^{e}
      (g) —C(═O)—NR^{e}R^{e′}
      (h) —O—C(═O)—R^{e}
      (i) —S(O)_{y}R^{e},
      (j) —S(O)_{y}NR^{e}R^{e′},
      (k) —S(O)_{y}NR^{e}—C(═O)C₁₋₆alkyl, wherein said alkyl is unsubstituted or substituted with one or more halogen,
      (l) —NR^{e}R^{e′}, and
      (m) —NR^{e}—C(═O)—R^{e′}, and R^{e} and R^{e′} are independently selected from the group consisting of
         (i) hydrogen,
         (ii) —C₁₋₁₀ alkyl,
         (iii) —C₂₋₁₀ alkenyl,
         (iv) —C₂₋₁₀ alkynyl,
         (v) a carbocyclic group having from 3 to 8 ring carbon atoms, optionally having from one to three ring carbon atoms replaced with S, N, C(═O) or O,
         (vi) —C₀₋₁₀ alkyl-C₆₋₁₀ aryl, and
         (vii) a heteroaryl group having from 5-10 ring atoms;
or R⁵ is hydrogen and R⁴ and R⁶ are linked together to form a carbocyclic group having from 3 to 8 ring carbon atoms, optionally having a single carbon-carbon double bond, and optionally having from one to three ring carbon atoms replaced with S, N, C(═O) or O,
   said carbocyclic group unsubstituted or substituted with one or more
      (a) hydroxyl,
      (b) —NR^{f}R^{f′},
      (c) —C(═O)—O—R^{f},
      (d) —OPO₃,
      wherein R^{f} is selected from the group consisting of
         (i) hydrogen, and
         (ii) —C₁₋₆ alkyl;
and pharmaceutically acceptable salts thereof.

In one embodiment, X¹ and X² are both —O—. In another embodiment, X¹ is —O— and X² is —NH—, or X¹ is —NH— and X² is —O—.

In certain embodiments, n is 0 and m is 1.

In certain embodiments, R⁵ and R⁶ are each hydrogen, and R⁴ is -Q-R^{a}.

In certain embodiments, the invention is directed to compounds of formula (I) having the structure of formula (I′): 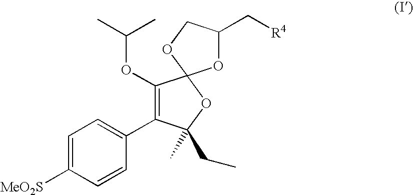
wherein R⁴ is as defined above, and pharmaceutically acceptable salts thereof.

In another embodiment, the invention is directed to imine compounds of formula (II): 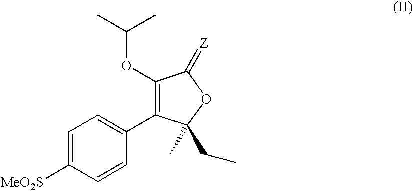
wherein Z is an amino acid or amino acid derivative which is linked to (II) at a nitrogen atom via an imine bond, and pharmaceutically acceptable salts thereof.

The imine compounds of formula (II) may also be present in tautomeric form as ortho-aminal compounds of formula (II′), as shown below: 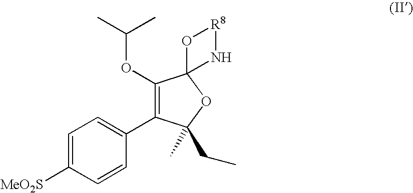
wherein R⁸ represents the amino acid or amino acid derivative moiety.

In preferred embodiments, Z is an amino acid selected from the group consisting of glycine, alanine, arginine, asparagine, aspartic acid, glutamic acid, cystine, glutamine, histidine, leucine, isoleucine, lysine, methionine, phenylalanine, proline, serine, threonine, tryptophan, tyrosine or valine. While either the D- or L-amino acid isomer may be used, L-amino acids are preferred.

When Z is an amino acid having more than one amine group (e.g., lysine, arginine), the point of attachment may be either at the primary amine group or at the secondary amine group (located on the amino acid side chain). When Z is lysine, the point of attachment is preferably at the primary amine.

Exemplary compounds of formula (II) include 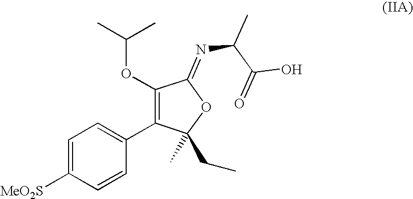
wherein Z is L-alanine; 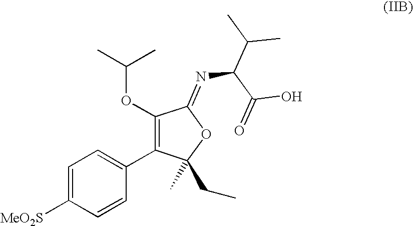
wherein Z is L-valine; and 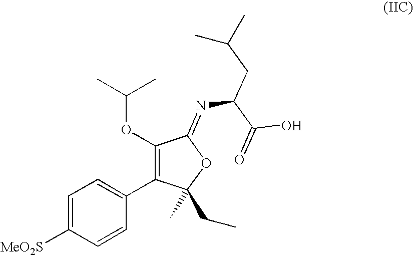
wherein Z is L-leucine. Each of IIA, IIB and IIC may also be present in its tautomeric ortho-aminal form.

An exemplary compound of formula (II) and its tautomer (II′), when Z is L-serine, is shown below:

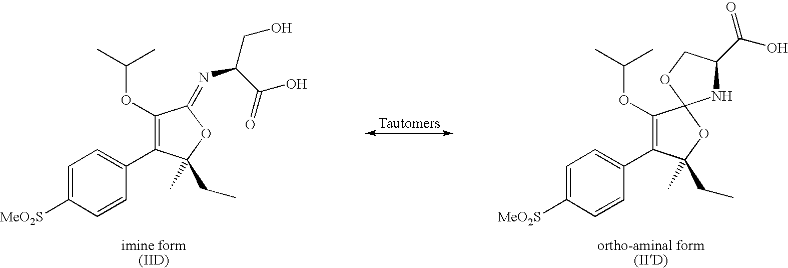

When Z is cysteine, the compound of formula (II) may be present in its imine form, or in its tautomeric form via the pendent thiol, as shown below:

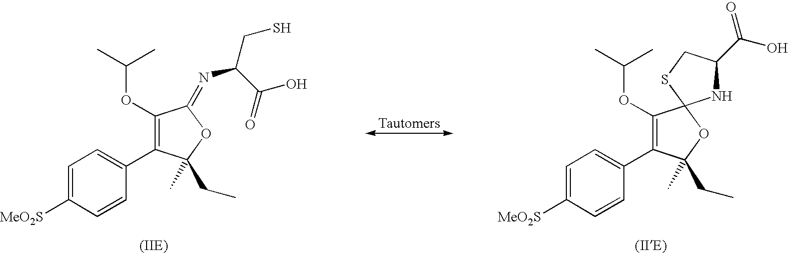

In another embodiment, the invention is directed to compounds of formula (III): 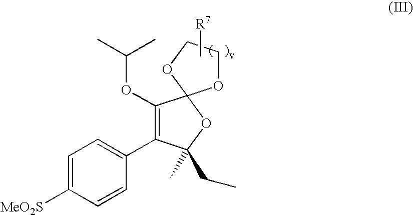
wherein R⁷ is a sugar molecule which is fused to remainder of compound (III), and v is 1 or 2.

Exemplary compounds of formula (III) include the following:

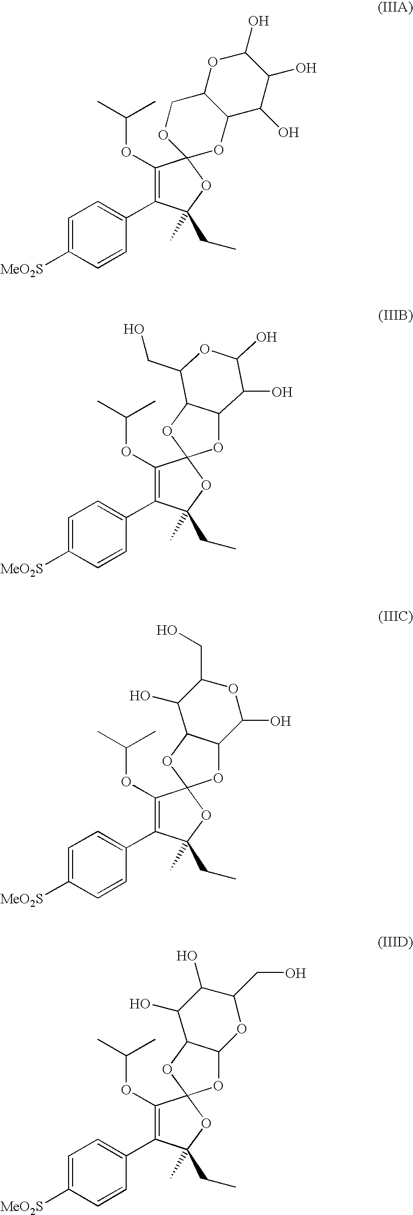

In certain embodiments, the compounds of the invention are selected from the Examples as described herein.

The compounds of the invention are prodrugs of selective COX-2 inhibitors, and exert their action by conversion in vivo to the active and selective COX-2 inhibitor 5(S)-ethyl-3-isopropoxy-4-(4-methanesulfonylphenyl)-5-methyl-5H-furan-2-one: 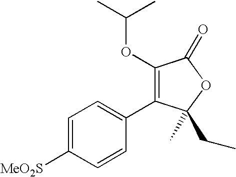
which is described in U.S. Pat. No. 6,020,343.

The compounds of the present invention have utility in treating, ameliorating or controlling stroke and the neurological injuries caused by stroke.

As used herein, the term “stroke” refers to a clinical event involving impairment of cerebral circulation, resulting in neurological injury. Typically, stroke is manifest by the abrupt onset of a focal neurological deficit. Stroke results from a rupture or obstruction (as by a thrombus or embolus) of an artery of the brain.

Thus in further aspects, the invention encompasses pharmaceutical compositions for treating stroke as defined above comprising a non-toxic therapeutically effective amount of a compound of the invention as defined above, and one or more ingredients such as other COX-2 inhibitors, antioxidants, nitric oxide synthase inhibitors, rho kinase inhibitors, angiotension II type-1 receptor antagonists, glycogen synthase kinase 3 inhibitors, sodium or calcium channel blockers, p38 MAP kinase inhibitors, thromboxane AX-synthetase inhibitors, statins, beta andrenergic blockers, NMDA receptor antagonists (including NR2B antagonists), 5-HT_{1A} agonists, platelet fibrinogen receptor antagonists, DPIV inhibitors, PDEIV antagonists, PPAR inhibitors, AMPA receptor antagonists, neurokinin inhibitors, bradykinin inhibitors, thrombin inhibitors, nociceptin antagonists, selective androgen receptor modulators and vasodilators.

In addition to stroke, by virtue of its in vivo conversion to a compound with high inhibitory activity against COX-2 and a specificity for COX-2 over COX-1, the compounds of the invention are useful in treatment of cyclooxygenase mediated diseases or disorders, including for the relief of pain (including neuropathic pain), fibromyalgia, fever and inflammation of a variety of conditions including rheumatic fever, symptoms associated with influenza or other viral infections, common cold, low back and neck pain, dysmenorrhea, headache; toothache, sprains and strains, myositis, neuralgia, synovitis, arthritis, including rheumatoid arthritis degenerative joint diseases (osteoarthritis), gout and ankylosing spondylitis, bursitis, bums, injuries, following surgical and dental procedures, and sepsis. In addition, the compounds of the invention may inhibit cellular neoplastic transformations and metastic tumor growth and hence can be used in the treatment of cancer. Compounds of the invention may also be useful for the treatment of traumatic brain injury, spinal cord injury, memory impairment, dementia (including vascular dementia, pre-senile and senile dementia, and in particular, dementia associated with Alzheimer's Disease).

The compounds of the invention will also prove useful as an alternative to conventional NSAID's, particularly where such non-steroidal antiinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anaemia such as hypoprothrombinemia, haemophilia or other bleeding problems; kidney disease; those prior to surgery or taking anticoagulants.

Thus in further aspects, the invention encompasses pharmaceutical compositions for treating cyclooxygenase-2 mediated diseases as defined above comprising a non-toxic therapeutically effective amount of a compound of the invention as defined above and one or more ingredients such as another pain reliever including acetaminophen or phenacetin; a potentiator including caffeine; an H2 antagonist, aluminum or magnesium hydroxide, simethicone, a decongestant including phenylephrine, phenylpropanolamine, pseudoephedrine, oxymetazoline, ephinephrine, naphazoline, xylometazoline, propylhexedrine, or levo-desoxyephedrine; an antitussive including codeine, hydrocodone, caramiphen, carbetapentane, or dextramethorphan; a diuretic; a sedating or non-sedating antihistamine. In addition the invention encompasses a method of treating cyclooxygenase mediated diseases comprising administration to a patient in need of such treatment a non-toxic therapeutically effect amount of the compound of the invention, optionally co-administered with one or more of such ingredients as listed immediately above.

As used herein, the term “ischemic stroke” refers to stroke characterized by localized tissue anemia due to obstruction of the inflow of arterial blood. Ischemic stroke is usually caused by atherothrombosis or embolism of a major cerebral artery, but may also be caused by coagulation disorders or nonatheromatous vascular disease.

The subject or patient to whom the compounds of the present invention is administered is generally a human being, male or female, in whom treatment of stroke is desired, but may also encompass other mammals, such as dogs, cats, mice, rats, cattle, horses, sheep, rabbits, monkeys, chimpanzees or other apes or primates, for which treatment of stroke is desired.

As used herein, the term “treatment” or “treating” means any administration of a compound of the present invention and includes (1) inhibiting stroke or the symptoms of stroke in an animal that is experiencing or displaying the pathology or symptomatology of stroke (i.e., arresting further development of the pathology and/or symptomatology, such as by enhancing plasticity of the stroke patient), or (2) ameliorating stroke or the symptoms of stroke in an animal that is experiencing or displaying the pathology or symptomatology of stroke (i.e., reversing the pathology and/or symptomatology). The term “controlling” includes preventing, treating, eradicating, ameliorating or otherwise reducing the severity of stroke, or reducing the risk of stroke.

In preferred embodiments, the invention is directed to compounds useful for the treatment of stroke. One class of patients to which a compound of the invention may be administered is a patient at risk for stroke. As used herein, the term “patient at risk for stroke” means an individual who has had a previous stroke, or has a risk factor for stroke. Known risk factors for stroke include atherosclerosis, arterial hypertension, lipohyalinosis, hyperlipidemia, hypercholesterolemia, atrial fibrillation, smoking, inflammatory markers (including C-reactive protein), infection, homocysteine, sleep-disordered breathing, cerebral autosomal dominant arteriopathy with subcortial infarcts and leuko-encephalopathy (CADASIL), migraine headaches, sickle-cell anemia, antiphospholipid antibody syndrome, arterial dissection, cocaine abuse and obesity.

Efforts at “controlling” stroke (including preventing stroke) can be divided into the primary prevention of stroke (treatment of patients who have not had any prior transient ischemic attacks or strokes, and have no neurological symptoms) and secondary prevention of stroke (treatment of patients who have had a prior transient ischemic attack or stroke). Primary prevention of stroke includes non-pharmacologic interventions, such as smoking cessation, healthy eating patterns, increased physical activity and weight management. Primary prevention also includes certain pharmacologic interventions, such as blood pressure control, treatment of atrial fibrillation, and management of diabetes, if appropriate. As part of the primary prevention of stroke, patients at high risk of coronary heart disease are often treated with aspirin. As part of primary prevention, patients having high amounts of low density lipoprotein (LDL) are often subject to blood lipid management, to reduce LDL levels to acceptable levels, e.g. below 160 mg/dl.

The secondary prevention of stroke often involves the same pharmacologic and non-pharmacologic interventions used for primary prevention, including blood pressure control, treatment of atrial fibrillation, management of diabetes, treatment with aspirin, and blood lipid management. Additional common secondary prevention interventions include the use of antiplatelet agents (such as clopidrogel), anticoagulants (such as warfarin), and anti-hypertension agents (such as beta andrenergic antagonists).

A second class of patients to which a compound of the invention may be administered are acute stroke patients, i.e., patients who have suffered ischemic stroke within the last 7 days. One preferred class of acute stroke patients are those who have suffered stroke within the last 3 days. A more preferred class of acute stroke patients are those who have suffered stroke within the last 48 hours, even more preferably within the last 24 hours. As common in the art of treating stroke, patients may be classified according to the period of time when stroke occurred. So, for example, one class of acute stroke patients are those who have suffered stroke within the last 18 hours. Another class of acute stroke patients are those who have suffered stroke within the last 12 hours. Another class of acute stroke patients are those who have suffered stroke within the last 8 hours. Another class of acute stroke patients are those who have suffered stroke within the last 6 hours. Another class of acute stroke patients are those who have suffered stroke within the last 4 hours. Another class of acute stroke patients are those who have suffered stroke within the last 3 hours.

Treatment of acute stroke, i.e. treatment during the cerebral event causing stroke and the 7 days thereafter, involve treatment with thrombolytics such as recombinant tissue plasminogen activator (rtPA). However, rtPA has only been approved for treatment of acute stroke for use within the first three hours after stroke. Another potential agent for treatment of acute stroke is the neuroprotectant edaravone, which has been approved in Japan.

During acute ischemic stroke, the arterial occlusion caused by the thrombus or embolus results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of the infarct. An area around the infracted core, known as the ischemic penumbra, suffers a delayed and less severe infarct. For example, during acute stroke the penumbra may have a reduction in blood flow of from about 20-40%.

Patients who have suffered stroke more than 24 hours previously often develop cerebral edema which typically occurs at one to five days after stroke. As used herein, the term “cerebral edema” refers to fluid collecting in brain tissue due to cellular swelling and the breakdown of the blood-brain barrier. Post-stroke cerebral edema may also involve the exuding of cerebrospinal fluid from ependymal lining, or the creation of an osmotic environment due to blood clots or tissue injury. The osmotic environment allows the movement of water into interstitial spaces. Post-stroke cerebral edema is often responsible for a worsening in the stroke patient's clinical status.

A third class of patients to which a compound of the present invention may be administered are patients who have suffered stroke more than 7 days previously, who are typically in need of restorative treatment (including enhancing plasticity).

In addition to stroke, by virtue of its in vivo conversion to a compound with high inhibitory activity against COX-2 and a specificity for COX-2 over COX-1, the compounds of the invention are useful in treatment of cyclooxygenase mediated diseases or disorders, including for the relief of pain (including post-operative pain and neuropathic pain), fibromyalgia, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, bums, injuries, following surgical and dental procedures, and sepsis. In addition, the compounds of the invention may inhibit cellular neoplastic transformations and metastic tumor growth and hence can be used in the treatment of cancer. Compounds of the invention may also be useful for the treatment of traumatic brain injury, spinal cord injury, memory impairment, and dementia (including vascular dementia, pre-senile and senile dementia, and in particular, dementia associated with Alzheimer's Disease).

The compounds of the invention will also prove useful as an alternative to conventional NSAID'S, particularly where such non-steroidal antiinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anaemia such as hypoprothrombinemia, haemophilia or other bleeding problems; kidney disease; those prior to surgery or taking anticoagulants.

Thus in further aspects, the invention encompasses pharmaceutical compositions for treating cyclooxygenase-2 mediated diseases as defined above comprising a non-toxic therapeutically effective amount of the compounds of the invention as defined above and one or more ingredients such as another pain reliever including acetaminophen or phenacetin; a potentiator including caffeine; an H2 antagonist, aluminum or magnesium hydroxide, simethicone, a decongestant including phenylephrine, phenylpropanolamine, pseudophedrine, oxymetazoline, ephinephrine, naphazoline, xylometazoline, propylhexedrine, or levo-desoxyephedrine; an antiitussive including codeine, hydrocodone, caramiphen, carbetapentane, or dextramethorphan; a diuretic; a sedating or non-sedating antihistamine. In addition the invention encompasses a method of treating cyclooxygenase mediated diseases comprising administration to a patient in need of such treatment a non-toxic therapeutically effect amount of the compound of formula I, optionally co-administered with one or more of such ingredients as listed immediately above.

The invention is also directed to a method for the manufacture of a medicament or a composition for treating stroke or other COX-2 mediated diseases, comprising combining a compound of the present invention of with a pharmaceutical carrier or diluent.

As used herein, the term “tautomer” refers to a compound which exists in an equilibrium mixture and which can be isolated in either form and react through either form. The tautomers may differ in linkage, bond, or connections between atoms, and the position or distribution of the atoms in the molecule. In the context of this invention, compounds of formula (II) may be present in the imine form depicted above, or in the tautomeric ortho aminal form (II′), as shown below:

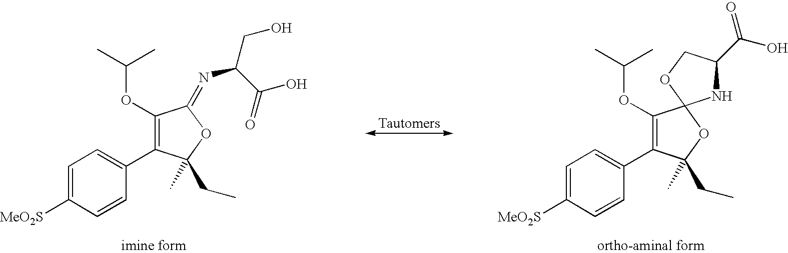

As used herein, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like.

As used herein, the term “alkoxy,” by itself or as part of another substituent, means the group —O— alkyl, wherein alkyl is defined above, having the number of carbon atoms designated (e.g., C₁₋₁₀ alkoxy means an alkoxy group having from one to ten carbon atoms). Preferred alkoxy groups for use in the invention are C₁₋₆ alkoxy groups, having from one to six carbon atoms. Exemplary preferred alkoxy groups include methoxy, ethoxy, propoxy, butoxy, sec-butoxy and pentoxy. Especially preferred alkoxy groups are C₁₋₃ alkoxy.

As used herein, the term “alkenyl,” by itself or as part of another substituent, means a straight or branched chain hydrocarbon radical having a single carbon-carbon double bond and the number of carbon atoms designated (e.g., C₂₋₁₀ alkenyl means an alkenyl group having from two to ten carbon atoms). Preferred alkenyl groups for use in the invention are C₂₋₆ alkenyl groups, having from two to six carbon atoms. Exemplary alkenyl groups include ethenyl and propenyl.

As used herein, the term “alkynyl,” by itself or as part of another substituent, means a straight or branched chain hydrocarbon radical having a single carbon-carbon triple bond and the number of carbon atoms designated (e.g., C₂₋₁₀ alkynyl means an alkynyl group having from two to ten carbon atoms). Preferred alkynyl groups for use in the invention are C₂₋₆ alkynyl groups, having from two to six carbon atoms. Exemplary alkynyl groups include ethynyl and propynyl.

As used herein, the term “cycloalkyl,” by itself or as part of another substituent, means a saturated cyclic hydrocarbon radical having the number of carbon atoms designated (e.g., C₃₋₁₂ cycloalkyl means a cycloalkyl group having from three to twelve carbon atoms). The term cycloalkyl as used herein includes mono-, bi- and tricyclic saturated carbocycles, as well as bridged and fused ring carbocycles, such as spiro fused ring systems.

Preferred cycloalkyl groups for use in the invention are monocyclic C₃₋₈ cycloalkyl groups, having from three to eight carbon atoms. Exemplary monocyclic cycloalkyl groups include cyclopropyl, cyclobutyl, cyclopentyl, cyclohexyl and the like. Exemplary bridged cycloalkyl groups include adamantly and norbornyl. Exemplary fused cycloalkyl groups include decahydronaphthalene.

As used herein, the term “carbocyclic,” by itself or as part of another substituent, means a cycloalkyl group as defined above, or a non-aromatic heterocyclic group. A non-aromatic heterocyclic group, by itself or as part of another substituent, means a cycloalkyl group as defined above in which one or more of the ring carbon atoms is replaced with a heteroatom (such as S, N or O), or a —C(═O)— group. Suitable non-aromatic heterocyclic groups for use in the invention include piperidinyl, piperazinyl, morpholinyl, tetrahydrofuranyl, tetrahydrothienyl, pyrrolidinyl, pyrazolidinyl and imidazolildinyl.

When a non-aromatic heterocyclic group as defined herein is substituted, the substituent may be bonded to a ring carbon atom of the heterocyclic group, or to a ring heteroatom (i.e., a nitrogen, oxygen or sulfur), which has a valence which permits substitution. Preferably, the substituent is bonded to a ring carbon atom. Similarly, when a non-aromatic heterocyclic group is defined as a substituent herein, the point of attachment may be at a ring carbon atom of the heterocyclic group, or on a ring heteroatom (i.e., a nitrogen, oxygen or sulfur), which has a valence which permits attachment. Preferably, the attachment is at a ring carbon atom.

As used herein, the term “aryl,” by itself or as part of another substituent, means an aromatic or cyclic radical having the number of carbon atoms designated (e.g., C₆₋₁₀ aryl means an aryl group having from six to ten carbons atoms). The term “aryl” includes multiple ring systems as well as single ring systems. Preferred aryl groups for use in the invention include phenyl and naphthyl.

The term “halo” or “halogen” includes fluoro, chloro, bromo and iodo.

As used herein, the term “heteroaryl,” by itself or as part of another substituent, means an aromatic cyclic group having at least one ring heteroatom (O, N or S). The term “heteroaryl” includes multiple ring systems as well as single ring systems. Exemplary heteroaryl groups for use in the invention include furyl, pyranyl, benzofuranyl, isobenzofuranyl, chromenyl, thienyl, benzothiophenyl, pyrrolyl, pyrazolyl, imidazolyl, pyridyl,pyrazinyl, pyrimidinyl, pyridazinyl, indolyl, indazolyl, benzimidazolyl, quinolyl, tetrazolyl and isoquinolyl.

When a heteroaryl group as defined herein is substituted, the substituent may be bonded to a ring carbon atom of the heteroaryl group, or to a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom (i.e., a nitrogen, oxygen or sulfur), which has a valence which permits attachment. Preferably, the attachment is at a ring carbon atom.

As used herein, the term “amino acid” refers to any of the naturally occurring amino acids which serve as the the units of peptides and proteins. Suitable amino acids include monoaminomonocarboxylic acid amino acids, of general structure NH₂—R—COOH, such as glycine (NH₂—COOH), alanine (NH₂CH(CH₃)—COOH), valine (NH₂CH—COOHCH(CH₃)₂), leucine (NH₂CH—COOHCH₂—CH(CH₃)₂), isovaline (NH₂C(CH₃)—COOHC(CH₃)₂), phenylalanine ((NH₂CH—COOHCH₂Ph), tyrosine ((NH₂CH—COOHCH₂PhOH), serine ((NH₂CH—COOHCH₂OH), cysteine ((NH₂CH—COOHCH₂SH), methionine ((NH₂CH—COOH(CH₂)₂S(CH₃)), isoleucine ((NH₂CH—COOHCH(CH₃)-Et), and threonine ((NH₂CH—COOHCHOH(CH₃)).

Other suitable amino acids include monoaminodicarboxylic acids, such as aspartic acid (NH₂CH—COOH(CH₂COOH) and glutamic acid ((NH₂CH—COOH(CH₂)₂—COOH), diaminomonocarboxylic acids, of general formula (NH2)2—R—COOH, such as arginine ((NH₂CH—COOH(CH₂)₃—NH—CNH(NH₂)), lysine ((NH2CH—COOH(CH₂)₄—NH₂), omithine ((NH₂CH—COOH(CH₂)₃—NH₂), asparagine ((NH₂CH—COOHCH₂—CONH₂), citrulline ((NH₂CH—COOH(CH₂)₃—NH—CONH₂), glutamine (NH₂CH—COOH(CH₂)₂—CONH₂), and heterocyclic amino acids, such as histidine: 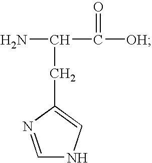
tryptophan: 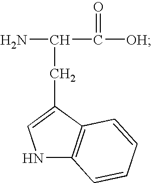
and proline:

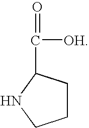

Some of the compounds of the instant invention have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of these compounds. Compounds described herein may contain one or more double bonds, and may thus give rise to cis/trans isomers as well as other conformational isomers. The present invention includes all such possible isomers as well as mixtures of such isomers. As used herein, the term “prodrug” refers to a molecule that is inert, i.e. not pharmacologically active, but that has pharmacological activity upon activation by a biological system. For example, a prodrug is a compound which is inert when in a tablet, capsule or other pharmaceutical composition, but is modified and becomes pharmacologically active in vivo, upon ingestion by a mammal. Thus, compounds of formulas (I), (II) and (III) which are modified in vivo to release compounds which are pharmacologically active in the treatment of stroke, are prodrugs.

### Methods of Synthesis

The compounds of the invention are derived from the compound 5(S)-ethyl-3-isopropoxy-4-(4-methanesulfonylphenyl)-5-methyl-5H-furan-2-one, shown below: 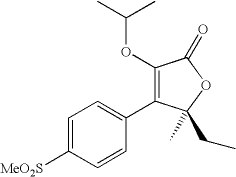
which is described in U.S. Pat. No. 6,020,343, as compound 144. The '343 patent is hereby incorporated by reference.

The compounds of the present invention can be prepared according to the following methods:

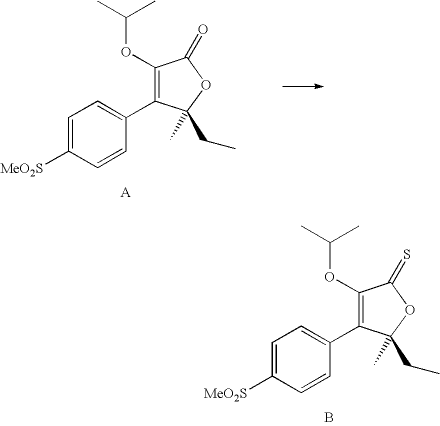

Compound A (5(S)-ethyl-3-isopropoxy-4-(4-methanesulfonylphenyl)-5-methyl-5H-furan-2-one) is converted to its corresponding thiolactone B by reflux of A with Lawesson's reagent in toluene. Compound B may then be converted to compounds of formula (II), such as imidate C, or compounds of formula (I), such as ortho ester D, as shown below: 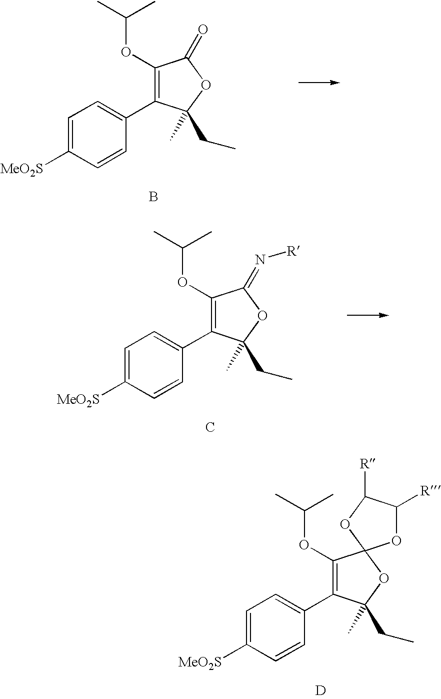
by a metal ion mediated desulfurization-condensation reaction between the thioester B and an amine or diol (for example, by reaction with AgOTf and Et₃N in the presence of an appropriate nucleophile).

Compounds of formula (III) may be formed by reaction of B with the desired sugar molecule, by a similar desulfurization-condensation reaction as used to form compounds of formula (I) or (II), followed by deprotection. Suitable protecting groups P for use in the synthesis include benzyl, acetate, trialkylsilyl or any other suitable protecting group or combination thereof, whereby removal is facile without compromising the orthoester functionality.

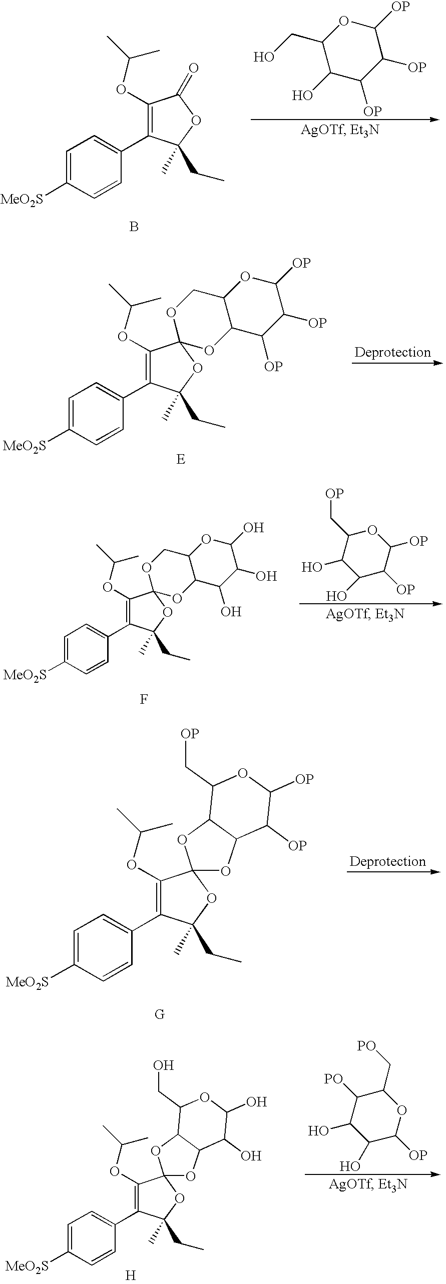

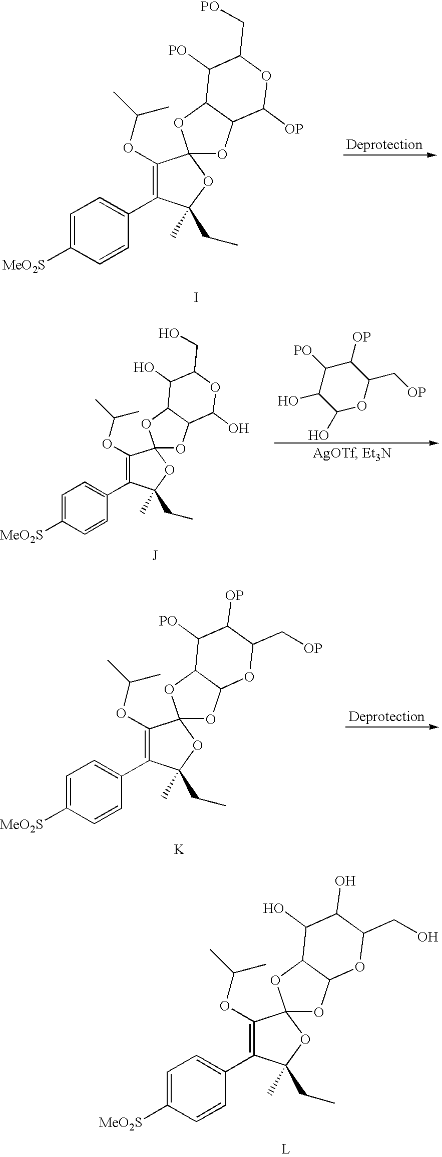

The compounds of formula (III) may be formed according to the above synthesis in a stereoselective manner by using an optically pure sugar molecule, or may be formed as racemates by using racemic sugar molecules.

By “pharmaceutically acceptable” it is meant the carrier, diluent or excipient must be compatible with the other ingredients of the formulation and not deleterious to the recipient thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, trifluoroacetic acid and the like.

The terms “administration of” or “administering a” compound should be understood to mean providing a compound of the invention to the individual in need of treatment in a form that can be introduced into that individual's body in a therapeutically useful form and therapeutically useful amount, including, but not limited to oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician. As used herein, the term “treatment” refers to the treatment of the mentioned conditions, particularly in a patient who demonstrates symptoms of the disease or disorder.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

The pharmaceutical compositions of the present invention encompass any composition made by admixing a compound of the present invention and a pharmaceutically acceptable carrier. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices.

Pharmaceutical compositions intended for oral use may be prepared according to any method known to the art for the manufacture of pharmaceutical compositions, and may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

Specific dosages of the compounds of the present invention for oral use, or pharmaceutically acceptable salts thereof, for administration include 1 mg, 5 mg, 10 mg, 30 mg, 80 mg, 100 mg, 150 mg, 300 mg and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions useful for treatment may comprise about 1 mg, 5 mg, 10 mg, 30 mg, 80 mg, 100 mg, 150 mg, 300 mg and 500 mg of active ingredient Pharmaceutical compositions of the invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of the invention can also be in a form suitable for rectal administration wherein the carrier is a solid. It is preferable that the mixture forms unit dose suppositories. Suitable carriers include cocoa butter and other materials commonly used in the art.

The invention is also directed to a therapeutically effective intravenous formulation of the compounds of the invention, which is solution stable and isotonic with human blood. The intravenous formulation preferably can be packaged in plastic or glass, and meets government and compendial (USP in the US) particulate standards, and can be used as effective therapy to treat stroke.

The intravenous formulation may contain a buffer which can maintain the pH of the intravenous formulation within a desirable range. The buffering agent also preferably acts as a complexing agent to maintain metal ions in solution which are leached out of the glass container. Both of these effects, maintaining the lower pH and complexing metal ions, prevents metal ions from precipitating and can maintain the intravenous formulation in an acceptable particulate profile for storage and subsequent use.

Pharmaceutical intravenous formulations of the invention will generally include a therapeutically effective amount of a compound of the invention to treat stroke, in addition to one or more pharmaceutically acceptable excipients. The compositions are advantageously prepared together with liquid inert carriers, such as water. Suitable liquid excipients/carriers are Water for Injection (US Pharmocoepia) and saline solution. The solution should be pyrogen-free, and also should be absent of particulate matter. Limits for the amount of particulate matter (i.e., extraneous, mobile undissolved substances, other than gas bubbles) which may be found in IV fluids are defined in the US Pharmacoepia.

Other suitable excipients and other additives for intravenous formulations include solvents such as ethanol, glycerol, propylene glycol, and mixtures thereof; stabilizers such as EDTA (ethylene diamine tetraacetic acid), citric acid, and mixtures thereof; antimicrobial preservatives, such as benzyl alcohol, methyl paraben, propyl paraben, and mixtures thereof; buffering agents, such as citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate, and mixtures thereof; tonicity modifiers, such as sodium chloride, mannitol, dextrose, and mixtures thereof; fluid and nutrient replenishers such as synthetic amino acids, dextrose, sodium chloride, sodium lactate, Ringer's solution, and other electrolyte solutions.

The buffer system is generally a mixture of a weak acid and a soluble salt thereof, e.g., sodium citrate/citric acid; or the monocation or dication salt of a dibasic acid, e.g., potassium hydrogen tartrate; sodium hydrogen tartrate, phosphoric acid/potassium dihydrogen phosphate, and phosphoric acid/disodium hydrogen phosphate. The amount of buffer system used is dependent on the desired pH and the amount of the compound of the invention. The choice of appropriate buffer and pH of a formulation, depending on solubility of the drug to be administered, is readily made by a person having ordinary skill in the art.

Intravenous formulations of compounds of the invention may be prepared according to procedures and dosing regimens known to those of ordinary skill in the art. For example, the compounds of the invention may be formulated in containers having a capacity of 100 to 1000 ml, for use in large volume injections administered by IV infusion. Alternatively, the compounds of the invention may be formulated in smaller or minitype infusion containers, having capacity for a 250 ml solution, or in containers having a capacity of 50 ml, 75 ml, 100 ml or 150 ml.

The doses to be administered parenterally are determined depending upon age, body weight, symptom, the desired therapeutic effect, the route of administration, and the duration of the treatment. In the human adult, the doses per person by parenteral administration (preferably intravenous administration) may be between 100 μg and 100 mg, up to several times per day. Particular ranges for dosage include from 0.01 to about 0.25 μg/kg/minute during a constant rate infusion, e.g., 0.15 μg/kg/minute. The amount of the compound of the invention administered must be determined based on the concentration of the parenteral formulation.

As mentioned above, the doses to be used depend upon various conditions. Therefore, there are cases in which doses lower than or greater than the ranges specified above may be used.

The following abbreviations are used throughout the text:
Me=methyl
MeOH=methanol
Et=ethyl
EtOAc=Ethyl Acetate
DMAP=4,4′-dimethoxytrityl chloride 4-dimethylamino pyridine
TEA: triethylamine
DMF: N,N′-dimethylformamide
rt=room temperature
hr=hour
min=minutes
M=molar

Examples 1 and 2 are formed according to the following scheme:

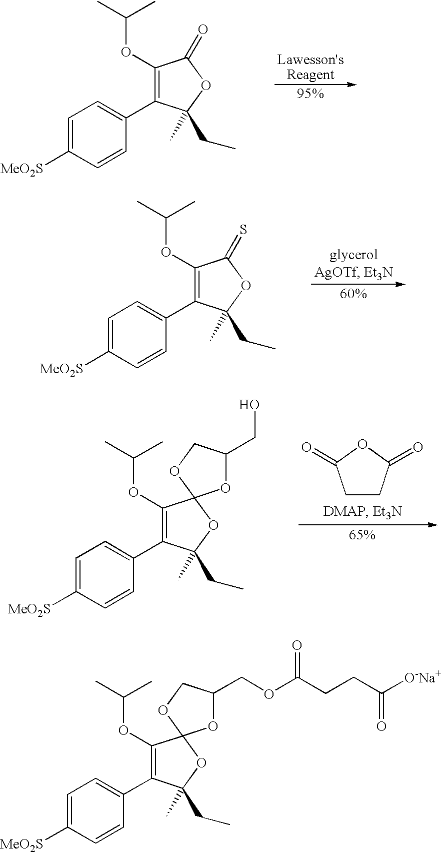

### Intermediate: 1: (5S)-5-ethyl-3-isopropoxy-5-methyl-4-[4-(methylsulfonyl)phenyl]furan-2(5H)-thione

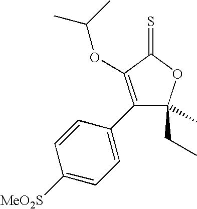

To a flask containing Lawesson's reagent (0.67 g, 1.66 mmol) and (5S)-5-ethyl-3-isopropoxy-5-methyl-4-[4-(methylsulfonyl)phenyl]furan-2(5H)-one (0.51 g, 1.51 mmol), dry toluene (15 mL) was added. The mixture was stirred vigorously under nitrogen. The flask was equipped with a condenser and the reaction was heated to reflux. After 18 hr, the reaction was allowed to cool to rt and then concentrated under vacuum. The crude material was purified by column chromatography with 20% EtOAc/hexanes to give the desired product as viscous oil that crystallized over time to give a yellow solid. ¹H-NMR (CDCl₃, 500 MHz) δ 7.71-7.69 (d, 2H), 7.42-7.39 (d, 2H), 5.32-5.25 (m, 1H), 2.24 (s, 3H), 1.71-1.66 (m, 1H), 1.43-1.36 (m, 1H), 1.16 (s, 3H), 1.06-1.05 (d, 3H), 1.02-1.00 (d, 3H), 0.61-0.58 (t, 3H). MS (ESI+ve) 355.5 (M+H)⁺.

### EXAMPLE 1

### {(7S)-7-ethyl-9-isopropoxy-7-methyl-8-[4-(methylsulfonyl)phenyl]-1,4,6-trioxaspiro[4.4]non-8-en-2-yl}methanol

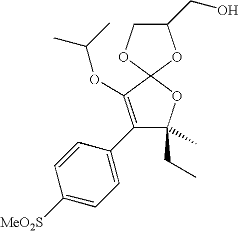

A solution of (5S)-5-ethyl-3-isopropoxy-5-methyl-4-[4-(methylsulfonyl)phenyl]furan-2(5H)-thione (0.20 g, 0.57 mmol), glycerol (0.06 g, 0.65 mmol), and silver triflate (0.36 g, 1.40 mmol) in dry acetonitrile (5 mL) was chilled to 0° C. in an ice bath under a nitrogen atmosphere. While stirring vigorously, triethylamine (0.32 mL, 2.28 mmol) was added dropwise to the solution. The reaction proceeded to give a black precipitate, and it was warmed to rt where it was stirred under nitrogen for 2 hr. The reaction mixture was filtered through Celite and the filtrate was concentrated under reduced pressure. The crude material was purified by column chromatography using a gradient of 10% EtOAc/hexanes to 60% EtOAc/hexanes over 30 min to give the desired product (mixture of diastereoisomers) as an oil that crystallized over time to give a white solid; ¹H-NMR (CDCl₃, 500 MHz) δ 7.73-7.69 (m, 1H), 7.34-7.32 (dd, 0.5 H), 7.20-7.18 (dd, 0.5 H), 4.29-4.25 (m 0.5 H), 4.15-3.85 (m, 3.5H), 3.71-3.63 (m, 1H), 3.31-3.28 (m, 0.5H), 3.11-3.08 (m, 0.5H), 2.23 (m, 3H), 1.64-1.45 (m, 2H), 1.42 (s, 1H), 1.35 (s, 1.5H), 1.31 (s, 1H), 1.09-1.06 (m, 3.5H), 0.98-0.91 (m, 4H); MS (ESI−ve) 413.5 (M−H)⁻.

### EXAMPLE 2

### Sodium 4-({(7S)-7-ethyl-9-isopropoxy-7-methyl-8-[4-(methylsulfonyl)phenyl]-1,4,6-trioxaspiro[4.4]non-8-en-2-yl}methoxy)-4-oxobutanoate

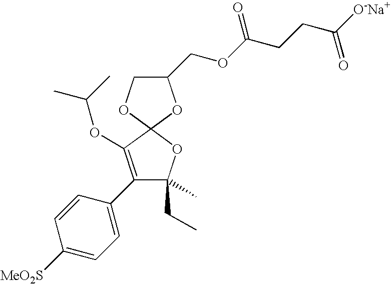

A solution of {(7S)-7-ethyl-9-isopropoxy-7-methyl-8-[4-(methylsulfonyl)phenyl]-1,4,6-trioxaspiro[4.4]non-8-en-2-yl}methanol, succinic anhydride and DMAP in dry dichloromethane was chilled to 0° C in. an ice bath. Triethylamine was then added dropwise to the solution, and the reaction was warmed to rt and stirred under nitrogen for 16 hr. The reaction was concentrated under reduced pressure and subsequently purified using a preparative HPLC instrument under basic conditions. ¹H-NMR (CDCl₃, 500 MHz) δ 8.00-7.97 (m, 2H), 7.73-7.70 (m, 2H), 4.59-4.57 (m, 0.5 H), 4.51-4.45 (m, 0.5H), 4.35-4.29 (m, 0.5H), 4.28-4.13 (m, 3.5H), 3.95-3.91 (m, 0.5H), 3.89-3.85 (m, 0.5H), 3.28 (m, 3H), 2.59-2.54 (m, 2H), 2.52-2.47 (m, 2H), 1.68-1.63 (m, 2H), 1.42-1.37 (m, 4H), 1.15-1.11 (m, 8H), 0.88-0.81 (m, 3H); MS (ESI−ve) 511.14 (M−H)⁻.

### EXAMPLE 3

### 2-{[({(2R,7S)-7-ethyl-9-isopropoxy-7-methyl-8-[4-(methylsulfonyl)phenyl]-1,4,6-trioxaspiro[4.4]non-8-en-2-yl}methoxy)(hydroxy)phosphoryl]oxy}-N,N,N-trimethylethanaminium

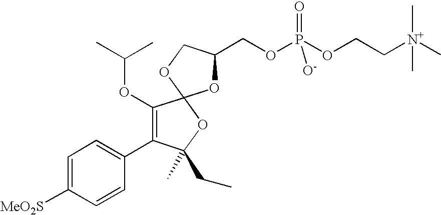

To a dry flask was added 1 eq of (5S)-5-ethyl-3-isopropoxy-5-methyl-4-[4-(methylsulfonyl)phenyl]furan-2(5H)-thone (200 mg, 0.564 mmol), 1.2 eq of n-Glycerophosphorylcholine (174 mg, 0.676 mmol), and 2.5 eq of silver triflate (362 mg, 1.410 mmol). The flask was purged with nitrogen and 5 mL of dry DMF was added. The reaction mixture was stirred for 2 h at rt (monitored by LC-MS), and quenched with 4 eq of TEA (314 μL, 2.26 mmol). The TEA was removed in vacuo, and the precipitate was filtered off and the filtrate purified without further concentration on silica gel (EtOAc, followed by 5% H₂O:MeCN, followed by 95% H₂O:MeCN) gave the desired compound. LC-MS calculated for C₂₄H₄₀NO₁₀PS 577, observed 578 (M+H)⁺. ¹H-NMR (500 MHz, MeOD) δ 7.69-8.09 (m, 4H), 4.92-4.93 (m, 1H), 4.76 (s, 3H), 4.32-4.33 (m, 2H), 3.94-4.06 (m, 2H), 3.85-3.89 (m, 1H), 3.61-3.69 (m, 4H), 3.32-3.33 (m, 3H), 3.21-3.26 (m, 9H), 1.98-2.13 (m, 2H), 1.66 (s, 3H), 1.24-1.27 (m, 3H), 0.802-0.831 (m, 3H).

### EXAMPLE 4

### N-[(5S)-5-ethyl-3-isopropoxy-5-methyl-4-[4-(methylsulfonyl)phenyl]furan-2(5H)-ylidene]-L-serine potassium salt

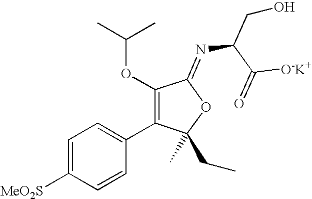 To a dry flask was added (5S)-5-ethyl-3-isopropoxy-5-methyl-4-[4-(methylsulfonyl)phenyl]furan-2(5H)-thone (200 mg, 0.56 mmol), L-serine methylester (100 mg, 0.62 mmol), and silver triflate (360 mg, 1.41 mmol). The flask was purged with nitrogen and 2 mL of dry MeCN was added followed by dropwise addition of Et₃N (0.25 mL). After 30 min, TLC analysis showed complete reaction and the solution was evaporated to dryness and the residue purified on silica gel (0 to 65% EtOAc in hexanes) to afford the L-serine methyl ester conjugate as a colorless foam. LC-MS calculated for C₂₁H₂₉NO₇S 439.52, observed 440.11 (M+H)⁺: ¹H NMR (500 MHz, C₆D₆) δ 7.75 (d, 2H), 7.52 (d, 2H), 5.62 (m, 1H), 4.72 (m, 1H), 4.09 (m, 1H), 3.37 (s, 3H), 2.64 (m, 1H), 2.62 (s, 3H), 1.65 (m, 2H), 1.53 (m, 1H), 1.18 (s, 3H), 1.20 (m, 6H), 0.77 (m, 4H). To this product (175 mg, 0.40 mmol) in dry THF (2 mL) was added potassium trimethylsilanolate (90% tech grade; 57 mg, 0.40 mmol) and stirred at rt. After 30 minutes a further 5 mg of KOTMS was added and TLC analysis indicated complete reaction. The solution was diluted with anhydrous ether and the resultant precipitate filtered, washed with ether and dried in vacuo to afford the desired title compound as a white solid. LC-MS calculated for C₂₀H₂₇NO₇S 425.15, observed 424.24 (M−H)⁻; ¹H NMR (500 MHz, D₂O) δ 8.09 (d, 2H), 7.90 (d, 2H), 4.45 (m, 1H), 4.35 (m, 1H), 3.98 (m, 1H), 3.87 (m, 1H), 3.80 (m, 1H), 3.35 (s, 3H), 1.95-1.85 (m, 2H), 1.55 (s, 3H), 1.20 (m, 6H), 0.88 (t, 3H).

### EXAMPLE 5

### (5R-5-[(2S,7R)-7-ethyl-7-methyl-9-(1-methylethoxy)-8-[4-(methylsulfonyl)phenyl]-1,4,6-trioxaspiro[4,4]non-8-en-2-yl]-3,4-dihydroxy-2(5H)-furanone monosodium salt

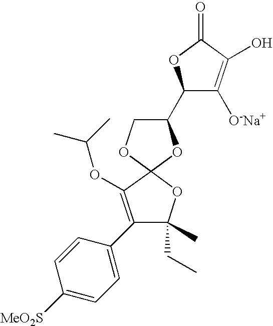 A solution of (5R)-5-ethyl-3-isopropoxy-5-methyl-4-[4-(methylsulfonyl)phenyl]furan-2(5H)-thione (0.26 g, 0.73 mmol), dibenzyl ascorbate (0.27 g, 0.76 mmol), and silver triflate (0.45 g, 1.75 mmol) in dry acetonitrile (5 mL) was chilled to 0° C. in an ice bath under a nitrogen atmosphere. While stirring vigorously, triethylamine (0.40 mL, 2.87 mmol) was added dropwise to the solution. The reaction proceeded to give a black precipitate, and it was warmed to room temperature where it was stirred under nitrogen for 2 hr. The reaction mixture was filtered through Celite and the filtrate was concentrated under pressure. The crude material was purified by column chromatography using a gradient of 5% EtOAc/hexanes to 60% EtOAc/hexanes over 30 min to give the desired products (mixtures of diastereomers) as an oil (0.39 g, 79%). MS (ESI) 675.8 (Me⁻). This ortho-ester derivative (0.35 g, 0.51 mmol), was dissolved in 5% pyridine in dry MeOH and then a catalytic amount of Pd on C (20 mg) was added to the solution. Using a hydrogen filled balloon, the reaction was evacuated and then purged with hydrogen gas three times. The reaction was stirred under hydrogen overnight. The mixture was filtered through Celite and concentrated. The crude material was redissolved in THF, and 1 eq of 1 M NaOH was added. The solution was stirred for 0.5 hr, and then concentrated under vacuum. The resulting white solid was triturated with diethyl ether three times, filtered, and dried to afford the desired compound. MS (ESI) 495.1 (Me⁻).

The following compound was prepared in an analogous manner to that described in the Examples above, using methods A-D as described above.

| Example | Reagents and Conditions | Structure |
|---|---|---|
| 6 | i) H₂NOH, AgOTf, Et₃N, MeCN ii) 2-bromoacetic acid | 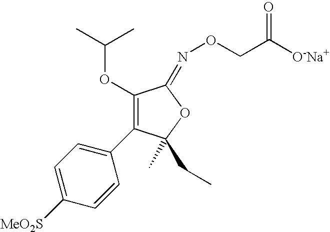 |

### EXAMPLE 6

### [[[(5S)-5-ethyl-5-methyl-3-(1-methylethoxy)-4-[4-(methylsulfonyl)phenyl]-2(5H)-furanylidene]amino]oxy]acetic acid sodium salt

While some of the compounds depicted as Examples above are represented in their basic form, the invention is intended to encompass both the salt and free acid forms of the compounds described above.

The physiochemical properties of Examples 1-6 and their ability to behave as pro-drugs and release in vivo the parent drug (the desired COX-2 inhibitor 5(S)-ethyl-3-isopropoxy-4-(4-methanesulfonylphenyl)-5-methyl-5H-furan-2-one) were examined. Results are shown below in Table 1. Examples 3-6 exhibited good solubility in water of >10 mg/mL, while 1 and 2 showed reduced solubility of <1 and 2 mg/mL respectively. Stability in solution was determined by HPLC analysis, and all of the pro-drugs were stable after 24 hr in water, with the exception of 5, which demonstrated rapid decomposition. To ascertain the rates of conversion of the pro-drugs in vivo, the compounds were dosed as a saline solution of the salt form (except for 1, which was dosed in a PEG-400 solution) in Sprague-Dawley rats at 2 mg/kg intravenously. Plasma levels were measured for levels of the respective pro-drug and the desired COX-2 inhibitor at 5, 15, 30, and 60 min time points. The plasma levels measured at 30 minutes are given below (Table 1).

TABLE 1
Physicochemical Properties of Prodrugs of the Invention

| | | | Levels of Desired | |
|---|---|---|---|---|
| | Solubility | | COX-2 Inhibitor | Levels of Pro-Drug @ |
| Ex | (mg/mL)^{a} | Stability^{b} | @ 0.5 hr^{c} (uM) | 0.5 hr^{d} (uM) |
| 1 | <1 | Y | 2.8^{d} | 1.4^{d} |
| 2 | 2 | Y | 0.4 | <0.1 |
| 3 | >20 | Y | 0.2 | 0.2 |
| 4 | >20 | Y | <0.1 | ND |
| 5 | >20 | N | 0.1 | ND |
| 6 | 10 | Y | <0.1 | ND |

^{a}Solubility is measured in deionized water @ 25° C.
^{b}4 mg/mL solution in water. Stability judged @ 24 hr by LC-MS
^{c}Concentration of 2 following a 2 mg/kg i.v. dose of pro-drug in Sprague-Dawley rats (n = 2); measuring at 5, 15, 30, 60 min. Value quoted is @ 30 min.
^{d}Dosed in PEG-400 solution

As can be ascertained from Table 1, imidates 4 and 6 failed to give appreciable amounts of the parent drug, yet the levels of each pro-drug dropped rapidly in vivo. Apparently, in vivo conversion of 4 and 6 was poor which suggests that the imidate derivatives are stable in vivo and are cleared rapidly as the pro-drug in the rat model.

Ortho-esters 1,2,3 and 5 demonstrated superior pharmacokinetic characteristics. Of the compounds that were dosed at 2 mg/kg, only compounds 1 and 2 converted to yield appreciable levels of the parent drug in the plasma at 30 min. Pro-drug 2 reached 0.4 μM in the plasma and the less soluble derivative 1 was measured at 2.8 μM. In comparison, the COX-2 IC₅₀ concentration of the parent drug is 0.4 μM in human whole blood.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What is claimed is:

1. A compound of formula (I) 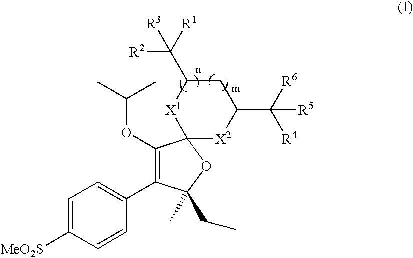
wherein:
X¹ and X² are selected from the group consisting of O, N and S;
n is 0 and m is 1, 2 or 3, or n is 1 and m is 0, 1 or 2;
R¹ and R⁴ are independently selected from the group consisting of
(1) -Q-R^{a},
(2) hydroxyl,
(3) a carbocyclic group having from 3 to 8 ring carbon atoms, optionally having from one to three ring carbon atoms replaced with S, N, C(═O) or O,
(4) —C₆₋₁₀ aryl, and
(5) a heteroaryl group having from 5-10 ring atoms,
wherein said carbocyclic group, aryl and heteroaryl are unsubstituted or substituted with one or more
(a) halogen,
(b) cyano,
(c) —NO₂,
(d) —C₁₋₆ alkyl, wherein said alkyl is unsubstituted or substituted with one or more halogen,
(e) —C₁₋₆ alkoxy,
(f) —C(═O)—(O)_{z}—R^{b},
(g) —C(═O)—NR^{b}R^{b′},
(h) —O—C(═O)—R^{b},
(i) —S(O)_{y}R^{b},
(j) —S(O)_{y}NR^{b}R^{b′},
(k) —S(O)_{y}NR^{b}—C(═O)—C₁₋₆ alkyl, wherein said alkyl is unsubstituted or substituted with one or more halogen,
(l) —NR^{b}R^{b′},
(m) —NR^{b}—C(═O)—R^{b′}, and
y is 0, 1 or 2
z is 0 or 1;
Q is selected from the group consisting of
(a) —O—,
(b) —O—C(═O)—,
(c) —S—,
(d) —SO₂—,
(e) —NR^{b},
(f) —NR^{b}—C(═O)—, and
(g) —O—PO₃—;
R^{a}, R^{b} and R^{b′} are independently selected from the group consisting of:
(i) —C₁₋₁₀ alkyl,
(ii) —C₂₋₁₀ alkenyl,
(iii) —C₂₋₁₀ alkynyl,
(iv) a carbocyclic group having from 3 to 8 ring carbon atoms, optionally having from one to three ring carbon atoms replaced with S, N, C(═O) or O, and
(v) —C₆₋₁₀ aryl,
wherein said carbocyclic group, alkyl, alkenyl, alkynyl and aryl are unsubstituted or substituted with one or more
(A) halogen,
(B) cyano,
(C) —NO₂,
(D) —C₁₋₆ alkyl, wherein said alkyl is unsubstituted or substituted with one or more halogen,
(E) —C₁₋₆alkoxy,
(F) —C(═O)—(O)_{z}—R^{c},
(G) —C(═O)—NR^{c}R^{c′},
(H) —O—C(═O)R^{c},
(I) —S(O)_{y}R^{c},
(J) —S(O)_{y}NR^{c}R^{c′},
(K) —S(O)_{y}NR—^{c}(═O)—C₁₋₆ alkyl, wherein said alkyl is unsubstituted or substituted with one or more halogen,
(L) —NR^{c}R^{c′}, and
(M) —NR^{c}—C(═O)R^{c′},
and R^{c} and R^{c′} are independently selected from the group consisting of
(1) hydrogen,
(2) —C₁₋₁₀ alkyl,
(3) —C₂₋₁₀ alkenyl,
(4) —C₂₋₁₀ alkynyl,
(5) a carbocyclic group having from 3 to 8 ring carbon atoms, optionally having from one to three ring carbon atoms replaced with S, N, C(═O) or O,
(6) —C₆₋₁₀ alkyl-C₆₋₁₀ aryl, and
(7) a heteroaryl group having from 5-10 ring atoms;
R², R³, R⁵ and R⁶ are independently selected from the group consisting of
(1) hydrogen,
(2) -Cl -l0 alkyl,
(3) -C₂-₁₀ alkenyl,
(4) C2-10 alkynyl, or
(5) <6-10 aryl,
wherein said alkyl, alkenyl, alkynyl, and aryl are unsubstituted or substituted with one or more
(a) halogen,
(b) cyano,
(c) —NO₂,
(d) —C₁₋₆ alkyl, wherein said alkyl is unsubstituted or substituted with one or more halogen,
(e) —C₁₋₆ alkoxy,
(f) —C(═O)—(O)_{z}—R^{e},
(g) —C(═O)—NR^{e}R^{e′,}
(h) —O—C(═O)—R^{e},
(i) —S(O)_{y}R^{e},
(j) —S(O)_{y}—NR^{e}R^{e′},
(k) —S(O)_{y}—NR^{e}—C(═O)—C₁₋₆ alkyl, wherein said alkyl is unsubstituted or substituted with one or more halogen,
(l) —NR^{e}R^{e′}, and
(m) —NR^{e}—C(═O)R^{e},
and R^{e} and R^{e′} are independently selected from the group consisting of
(i) hydrogen,
(ii) —C₁₋₁₀ alkyl,
(iii) —C₂₋₁₀ alkenyl,
(iv) —C₂₋₁₀ alkynyl,
(v) a carbocyclic group having from 3 to 8 ring carbon atoms, optionally having from one to three ring carbon atoms replaced with S, N, C(═O) or O,
(vi) —C₀₋₁₀ alkyl-C₆₋₁₀ aryl, and
(vii) a heteroaryl group having from 5-10 ring atoms;
or R⁵ is hydrogen and R⁴ and R⁶ are linked together to form a carbocyclic group having from 3 to 8 ring carbon atoms, optionally having a single carbon-carbon double bond, and optionally having from one to three ring carbon atoms replaced with S, N, C(═O) or O,
said carbocyclic group unsubstituted or substituted with one or more
(a) hydroxyl,
(b) —NR^{f}R^{f′},
(c) —C(═O)—O—R^{f},
(d) —OPO₃,
wherein R^{f} is selected from the group consisting of
(i) hydrogen, and
(ii) —C₁₋₆ alkyl,
and pharmaceutically acceptable salts thereof.

2. The compound of claim 1 wherein X¹ and X² are both —O—.

3. The compound of claim 1 wherein X¹ is —O— and X² is —NH—, or X¹ is —NH— and X² is —O—.

4. The compound of claim 1, wherein n is 0 and m is 1.

5. The compound of claim 1, wherein R⁵ and R⁶ are each hydrogen, and R⁴ is -Q-R^{a}.

6. The compound of claim 1 which is 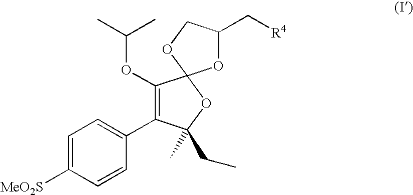
wherein R⁴ is as defined in claim 1, and pharmaceutically acceptable salts thereof.

7. A method of treating stroke, comprising administering a compound of claim 1 to a patient in need thereof.

8. The method of claim 7, wherein the patient is an acute stroke patient.

9. A compound of formula (II): 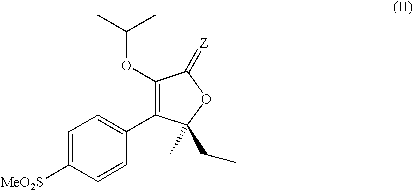
wherein Z is an amino acid or amino acid derivative which is linked to (II) at a nitrogen atom via an imine bond, and pharmaceutically acceptable salts thereof.

10. The compound of claim 9 wherein Z is an amino acid selected from the group consisting of glycine, alanine, arginine, asparagine, aspartic acid, glutamic acid, cystine, glutamine, histidine, leucine, isoleucine, lysine, methionine, phenylalanine, proline, serine, threonine, tryptophan, tyrosine or valine.

11. A method of treating stroke, comprising administering a compound of claim 6 to a patient in need thereof.

12. The method of claim 11, wherein the patient is an acute stroke patient.

13. A compound of formula (III): 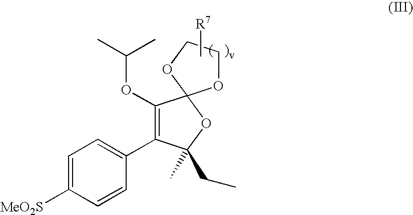
wherein R⁷ is a sugar molecule which is fused compound (III), and v is 1 or 2.

14. A method of treating stroke, comprising administering a compound of claim 13 to a patient in need thereof.

15. The method of claim 14, wherein the patient is an acute stroke patient.

16. A method of treating stroke, comprising administering a compound of claim 16 to a patient in need thereof.

17. A pharmaceutical composition suitable for intravenous administration, comprising a compound of claim 1, and a pharmaceutically acceptable carrier.

18. A pharmaceutical composition suitable for intravenous administration, comprising a compound of claim 9, and a pharmaceutically acceptable carrier.

19. A pharmaceutical composition suitable for intravenous administration, comprising a compound of claim 13 and a pharmaceutically acceptable carrier.
